(19) United States
(12) Patent Application Publication
(10) Pub. No.: US 2023/0398106 A1
(43) Pub. Date: Dec. 14, 2023

(54) COMBINATION THERAPY OF DONEPEZIL AND SILDENAFIL FOR THE TREATMENT OF ALZHEIMER'S DISEASE OR COGNITIVE IMPAIRMENT

(71) Applicant: NEURORIVE INC, Seoul (KR)

(72) Inventors: Sukchan LEE, Yongin-si (KR); Yong Ha CHI, Seogwipo-si (KR); Hyun-Hee RYU, Suwon-si (KR); Miso JEONG, Deajeon (KR)

(73) Assignee: NEURORIVE INC, Seoul (KR)

(21) Appl. No.: 18/035,403

(22) PCT Filed: Nov. 5, 2021
(86) PCT No.: PCT/KR2021/016080

§ 371 Date: May 4, 2023

(30) Foreign Application Priority Data
Nov. 5, 2020 (KR) 10-2020-0146677

(51) Int. Cl. A61K 31/445; A61K 31/519; A61P 25/28

(52) U.S. Cl. CPC A61K 31/445; A61K 31/519; A61P 25/28

(57) ABSTRACT

The present invention relates to a combination therapy of donepezil and sildenafil for the prevention, treatment or improvement of Alzheimer's disease or cognitive impairment. The combined administration of donepezil and sildenafil according to the present invention has a superior effect on improving Alzheimer's disease or cognitive impairment as compared to single administration of donepezil, which is able to be effectively used as a therapeutic therapy or a complex agent for Alzheimer's disease or cognitive impairment.

### TECHNICAL FIELD

The present invention relates to a combination therapy of donepezil and sildenafil for the treatment of Alzheimer's disease or cognitive impairment.

### BACKGROUND ART

Cognitive impairment refers to a problem that occurs in the process of thinking, which has a concept that includes loss of reasoning ability, forgetting, learning disability, concentration problem, decline in intelligence, and other reduction in mental functions. The cognitive impairment may be caused by a disease occurring during the development of the fetus, childbirth, immediately after childbirth, or at any point in life, and in some cases, the cause may not be identified, particularly in newborns or young children. Early causes of the cognitive impairment include chromosomal abnormalities and genetic syndromes, malnutrition, drug exposure before childbirth, contamination of heavy metals such as lead, hypoglycemia, neonatal jaundice, hypothyroidism, trauma or child abuse, low oxygen in the womb, dystocia, premature birth, and the like.

Dementia, a typical disease of cognitive impairment, is a pathological phenomenon that is distinct from normal aging, and is divided into Alzheimer's disease, vascular dementia, and other dementias caused by alcohol poisoning, trauma, and sequelae of Parkinson's disease, and the like, depending on the cause. Alzheimer's disease accounts for 50-70% of dementia-causing diseases.

Acetylcholine is a neurotransmitter that acts not only in the central nervous system but also in the peripheral nervous system. A low level of acetylcholine has been associated with diseases in which cognitive impairment plays a significant role, such as Alzheimer's disease. In fact, the administration of donepezil, an acetylcholinesterase inhibitor, is one of the main treatment paradigms for Alzheimer's disease. Donepezil is used for the treatment of mild to severe Alzheimer's disease, and may be administered from 5 mg to 23 mg per day depending on the severity of the disease. It is widely believed that cholinergic neurotransmission deficit is partly involved in the development of cognitive signs and symptoms shown in Alzheimer's disease, wherein it is presumed that donepezil exerts therapeutic efficacy by increasing the concentration of acetylcholine in the brain through reversible inhibition of acetylcholinesterase (AChE). However, donepezil is capable of being used only for treatment that slows down the progression of the disease, and there is no known treatment to cure Alzheimer's disease or to prevent or to stop disease progression. Further, donepezil does not help everyone suffering from Alzheimer's disease, and is not effective in many patients. Accordingly, there is a high unfulfilled need for more effective treatment for Alzheimer's disease or cognitive impairment symptoms and disease control/slowing therapy.

According to a recent review paper (2020), it is described that nitric oxide (NO), which has dual properties of neuroprotection and neurotoxicity in the central nervous system, is partially involved in Alzheimer's disease, and the activation of the NO signaling pathway may lead to phosphorylation of the transcription factor CREB (cAMP response element-binding protein) (i.e., NO/cGMP/PKG/CREB signaling pathway), thereby contributing to altered neuroplasticity and memory deficit alleviation in an animal model of Alzheimer's disease, and thus a PDE5 inhibitor is usable to treat memory disorders by activating the pathway (see Zuccarello, Elisa, et al. “Development of novel phosphodiesterase 5 inhibitors for the therapy of Alzheimer's disease.” Biochemical Pharmacology 176 (2020): 113818).

However, the specific combination and mixing ratio of donepezil and the PDE5 inhibitor capable of preventing, treating or improving symptoms of Alzheimer's disease or cognitive impairment are not known. Therefore, the present inventors confirmed that prevention, treatment or improvement effects of Alzheimer's disease or cognitive impairment could be exerted depending on a specific mixing ratio of sildenafil, which is one of the PDE5 inhibitors, and donepezil, and completed the present invention.

### DISCLOSURE OF INVENTION

### Technical Problem

An object of the present invention is to provide the use for the prevention or treatment of Alzheimer's disease or cognitive impairment according to combined administration of donepezil and sildenafil.

Another object of the present invention is to provide the use for the improvement of cognitive function in the combined administration of donepezil and sildenafil.

### Solution to Problem

The present inventors have studied and made efforts to achieve the above object, and as a result, confirmed that when donepezil and sildenafil were administered in combination, prevention or treatment effect on Alzheimer's disease or cognitive impairment was improved as compared to single administration of donepezil, and completed the present invention.

In one aspect, the present invention provides a composition for preventing, treating or improving Alzheimer's disease or cognitive impairment comprising: (i) donepezil or a pharmaceutically acceptable salt thereof; and (ii) sildenafil or a pharmaceutically acceptable salt thereof.

In the composition above, a weight ratio of (i) donepezil or the pharmaceutically acceptable salt thereof; and (ii) sildenafil or the pharmaceutically acceptable salt thereof may be 1:0.6 to 1:2.0.

In the composition above, (i) donepezil or the pharmaceutically acceptable salt thereof; and (ii) sildenafil or the pharmaceutically acceptable salt thereof may be administered simultaneously or at different times. In case the composition is administered simultaneously, the composition may be a combination formulation including (i) donepezil or a pharmaceutically acceptable salt thereof; and (ii) sildenafil or a pharmaceutically acceptable salt thereof.

In case the composition is a combination formulation, the combination formulation may include (i) 2.5 to 23 mg of donepezil or the pharmaceutically acceptable salt thereof; and (ii) 2.5 to 50 mg of sildenafil or the pharmaceutically acceptable salt thereof.

The above composition may be a pharmaceutical composition or a food composition.

In another aspect, the present invention provides a composition for improving cognitive function comprising: (i) donepezil or a pharmaceutically acceptable salt thereof; and (ii) sildenafil or a pharmaceutically acceptable salt thereof.

In the composition for improving cognitive function above, a weight ratio of (i) donepezil or the pharmaceutically acceptable salt thereof; and (ii) sildenafil or the pharmaceutically acceptable salt thereof may be 1:0.6 to 1:2.0.

In one aspect, the present invention provides a method for prevention, treatment or improvement of Alzheimer's disease or cognitive impairment, wherein the method comprises administering a therapeutically effective amounts of (i) donepezil or a pharmaceutically acceptable salt thereof; and (ii) sildenafil or a pharmaceutically acceptable salt thereof together into a patient.

In one aspect, the present invention provides a method for improvement of cognitive function, wherein the method comprises administering an effective amounts of (i) donepezil or a pharmaceutically acceptable salt thereof; and (ii) sildenafil or a pharmaceutically acceptable salt thereof together into a human.

### Advantageous Effects of Invention

The combined administration of donepezil and sildenafil according to the present invention has a superior effect on improving Alzheimer's disease or cognitive impairment as compared to single administration of donepezil, which is able to be effectively used as a preventive, improving or therapeutic therapy or a complex agent for Alzheimer's disease or cognitive impairment.

### BRIEF DESCRIPTION OF DRAWINGS

FIG. 1 shows results of long-term potentiation (LTP) experiment after combined treatment of donepezil and sildenafil in an animal model of Alzheimer's disease or cognitive impairment; and

FIGS. 2A and 2B show results of the Morris water maze experiment after the combined administration of donepezil and sildenafil in an animal model of Alzheimer's disease or cognitive impairment.

### BEST MODE FOR CARRYING OUT THE INVENTION

Hereinafter, the present invention will be described in detail as exemplary embodiments of the present invention with reference to the accompanying drawings. However, the following exemplary embodiments are presented as examples of the present invention, and in a case where it is determined that detailed descriptions of a technique or configuration well known to those skilled in the art may unnecessarily obscure the gist of the present invention, detailed descriptions thereof may be omitted, and thus the present invention is not limited thereto. The present invention may be variously modified and applied within the description of the claims to be described below and within the equivalent scope interpreted therefrom.

In addition, terminologies used in the present specification are terms used to appropriately express preferred embodiments of the present invention, which may vary depending on the intention of users or operators, or customs in the field to which the present invention belongs, and the like. Accordingly, definitions of these terms should be established based on the contents throughout the present specification. Throughout the specification, when a part “includes” a component, it means that the part may further include other components rather than excluding the other components unless otherwise specified.

All technical terms used in the present invention, unless otherwise defined, are used in the same meaning as those of ordinary skill in the art generally understand in the related field of the present invention. In addition, preferred methods or samples are described in the present specification, but those similar thereto or the equivalents thereof are included in the scope of the present invention. Contents of all publications described in the present specification by reference are incorporated into the present invention.

The present inventors confirmed that the treatment effect on Alzheimer's disease or cognitive impairment was improved when donepezil and sildenafil were administered in combination as compared to donepezil single administration, and completed the present invention.

Accordingly, the present invention provides a composition for preventing, treating or improving Alzheimer's disease or a cognitive impairment disease, including (i) donepezil or a pharmaceutically acceptable salt thereof; and (ii) sildenafil or a pharmaceutically acceptable salt thereof.

In the present invention, the term donepezil is a compound represented by the following Chemical Formula 1:

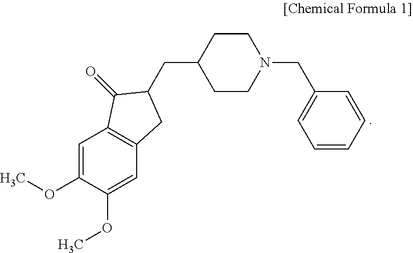 [SMILES: COc1cc2c(cc1OC)C(=O)C(CC1CCN(Cc3ccccc3)CC1)C2]

Donepezil or the pharmaceutically acceptable salt thereof is an acetylcholinesterase inhibitor (ACEi), which is known to be usable for the treatment of Alzheimer's dementia symptoms by oral administration, if based on donepezil hydrochloride, at 5 to 23 mg once a day.

In the present invention, the term sildenafil is a compound represented by the following Chemical Formula 2:

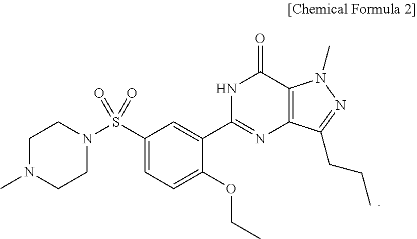 [SMILES: CCCc1nn(C)c2c(=O)[nH]c(-c3cc(S(=O)(=O)N4CCN(C)CC4)ccc3OCC)nc12]

Sildenafil or a pharmaceutically acceptable salt thereof is a phosphodiesterase 5 (PDE5) inhibitor, which is known to be used in the treatment of symptoms of erectile dysfunction when administered at a dose of 25-50 mg per day to males. In the present invention, sildenafil, as a PDE5 inhibitor, is distinguished in a mechanism way from drugs belonging to other competitive PDE inhibitors, such as PDE1 inhibitors (e.g., vinpocetine), PDE2 inhibitors (e.g., EHNA (erythro-9-(2-hydroxy-3-nonyl)adenine), BAY 60-7550 (2-[(3,4-dimethoxyphenyl)methyl]-7-[(1R)-1-hydroxyethyl]-4-phenylbutyl]-5-methyl-imidazo[5,1-f][1,2,4]triazine-4(1H)-one), oxindole, PDP (9-(6-phenyl-2-oxohex-3-yl)-2-(3,4-dimethoxybenzyl)-purin-6-one)), PDE3 inhibitors (e.g., anagrelide, cilostazol, enoximone, inamrinone, milrinone, pimobendan), PDE4 inhibitors (e.g., apremilast, drotaverine, ibudilast, luteolin, mesembrine, piclamilast, roflumilast, rolipram), PDE6 inhibitors, PDE7 inhibitors (e.g., quinazoline), PDE8 inhibitors, PDE9 inhibitors, PDE10 inhibitors (e.g., papaverine), PDE11 inhibitors, PDE12 inhibitors, or combinations thereof. Further, sildenafil is distinguished from PDE5 inhibitors having different chemical structures and functional properties from sildenafil, such as avanafil, dipyridamole, icariin, tadalafil, udenafil, and vardenafil.

In the present invention, Alzheimer's disease is a neurodegenerative disease that accounts for a major cause of dementia, and has the same meaning as commonly used in the art.

In the present invention, cognitive impairment refers to a problem that occurs in the process of thinking in humans, which has a concept that includes loss of reasoning ability, forgetting, learning disability, concentration problem, decline in intelligence, and other reduction of mental functions.

As used herein, the term pharmaceutically acceptable salt refers to any organic or inorganic addition salt at a concentration having an effective action that is relatively non-toxic and harmless to a patient, in which side effects caused by the salt do not reduce the beneficial efficacy of the pharmacologically active ingredient. The pharmaceutically acceptable salt includes a salt derived from a pharmaceutically acceptable acid or base. The acid that is capable of being used in the preparation of the pharmaceutically acceptable salt may be an inorganic acid or an organic acid. The inorganic acid may be, for example, hydrochloric acid, sulfuric acid, nitric acid, phosphoric acid, perchloric acid, hydrobromic acid, and the like, and the organic acid may be, for example, acetic acid, methanesulfonic acid, ethanesulfonic acid, p-toluenesulfonic acid, fumaric acid, maleic acid, malonic acid, phthalic acid, succinic acid, lactic acid, citric acid, gluconic acid, tartaric acid, salicylic acid, malic acid, oxalic acid, benzoic acid, embonic acid, aspartic acid, glutamic acid, and the like, but the present invention is not limited thereto. In addition, amino acid addition bases prepared using natural amino acids such as alanine, glycine, and the like, may also be included in the pharmaceutically acceptable salts of the present invention. Further, the base capable of being used in the preparation of the pharmaceutically acceptable salt may be, for example, tris(hydroxymethyl)methylamine, dicyclohexylamine, and the like, but is not limited thereto. For example, the pharmaceutically acceptable salt of donepezil may be donepezil hydrochloride, but is not limited thereto.

In the pharmaceutical composition of the present invention, the pharmaceutically acceptable salts of donepezil and/or sildenafil may also include hydrates and solvates of donepezil and/or sildenafil. The hydrate or solvate may be prepared in a crystallized or recrystallized form obtained by dissolving donepezil and/or sildenafil in a water-miscible solvent such as methanol, ethanol, acetone, or 1,4-dioxane and then adding a free acid or a free base, but the present invention is not limited to the preparation method thereof.

In the pharmaceutical composition for preventing or treating Alzheimer's disease of the present invention, a weight ratio of (i) donepezil or a pharmaceutically acceptable salt thereof; and (ii) sildenafil or a pharmaceutically acceptable salt thereof may be 1:0.6 to 1:2.0. More specifically, in the pharmaceutical composition for preventing or treating Alzheimer's disease of the present invention, the weight ratio of (i) donepezil or a pharmaceutically acceptable salt thereof; and (ii) sildenafil or a pharmaceutically acceptable salt thereof may be 1:0.6 or more, or 1:1 or more, and 1:2 or less. In an embodiment, the weight ratio of (i) donepezil or a pharmaceutically acceptable salt thereof; and (ii) sildenafil or a pharmaceutically acceptable salt thereof is about 1:1 to 1:1.5.

The molecular weight ratio of donepezil (379.5 g/mol) and sildenafil (474.58 g/mol) is about 1:1.25, and in the present specification, the molar ratio and the weight ratio may be used by mutually converting in consideration of the above ratio.

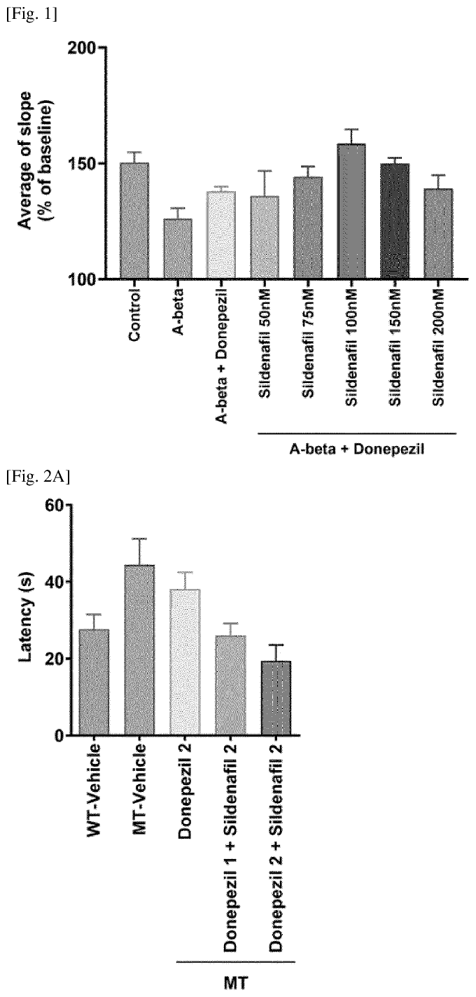

The present inventors confirmed that, as a result of treating the brain sections of the animal model of Alzheimer's disease or cognitive impairment with the combination of donepezil and sildenafil at various weight ratios, the change in synaptic plasticity was significantly improved as compared to the donepezil single treatment group. Specifically, the present inventors confirmed that the improvement effect on Alzheimer's disease or cognitive impairment was excellent in the range where the weight ratio of donepezil and sildenafil is about 1:0.6 to 1:2.0 (FIG. 1).

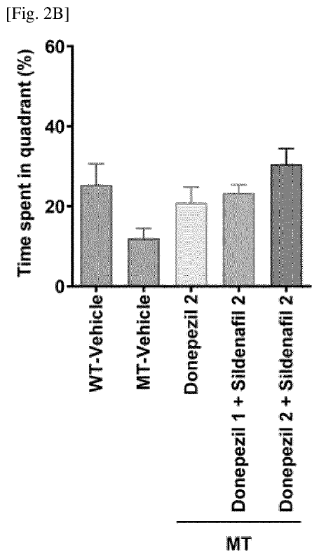

In addition, the present inventors confirmed that the behavioral indicators in the improvement of Alzheimer's disease or cognitive impairment were significantly improved when donepezil and sildenafil were administered in combination through the Morris water maze task, which is an animal behavioral experiment with Alzheimer's disease or cognitive impairment (FIGS. 2A, 2B).

Specifically, the present inventors confirmed from Examples of the present invention that when administering 1-2 mg/kg of donepezil and 2 mg/kg of sildenafil in combination to a model mouse of Alzheimer's disease or cognitive impairment, results of the Morris water maze experiment (FIGS. 2A and 2B) were significantly improved, and in particular, surprisingly confirmed that when 1 mg/kg of donepezil and 2 mg/kg of sildenafil were administered in combination to a disease model mouse, the Alzheimer's disease or cognitive impairment index was improved at a level similar to that of the group administered with 2 mg/kg of donepezil alone (FIGS. 2A and 2B).

Therefore, a desirable administration dose in humans may be converted in consideration of the optimal animal administration dose identified in the present invention, the mouse-human dose-response relationship, and No-observed-adverse-effect level (NOAEL), and the like. The dose conversion factor may be calculated by using the human equivalent dose (HED) described in document [FDA, US. “Guidance for Industry, Estimating the maximum safe starting dose in initial clinical trials for therapeutics in adult healthy volunteers.” FDA, ed (2005)]. The mouse-human dose conversion factor presented in the above document is 12.3, and when the dose (a) of the drug identified in the mouse is divided by the conversion factor, a/12.3 (mg/kg) dose is derived. Since pharmaceuticals are generally made based on an adult weighing 60 kg, when the value of a/12.3 (mg/kg) derived from above is multiplied by 60 kg, it is possible to prepare a pharmaceutical composition to be administered to humans at a dose of about 5×a (mg). Therefore, with reference to the optimal mouse dose ratio of donepezil and sildenafil confirmed in Examples of the present invention, a desirable dose ratio of the drug to be administered to humans may be derived.

By comprehensively considering the above mouse-human dose conversion formula and the dose of donepezil for which safety has been established, and the like, when the combined composition of donepezil and sildenafil of the present invention is provided as a combination formulation, the desirable weight of donepezil and sildenafil may be determined. In an embodiment, the weight of donepezil included in the combination formulation is 2.5 to 23 mg, and the weight of sildenafil is 2.5 to 50 mg.

The pharmaceutical composition of the present invention may be administered in a therapeutically effective amount. The therapeutically effective amount refers to a dosage of a drug exerting an effective prevention or treatment effect on Alzheimer's disease or cognitive impairment. A suitable total daily usage may be determined by the practitioner within the scope of correct medical judgment. It is preferred that the specific therapeutically effective amount for a particular patient will vary depending on various factors including the type and extent of the response to be achieved; the type and amount of drugs administered in combination; the specific composition whether other agents are used in some cases, the patient's age, weight, general health status, sex and diet, administration time, administration route, and treatment period, and similar factors well known in the medical field.

The pharmaceutical composition of the present invention may be administered orally or parenterally, preferably orally. In addition, the pharmaceutical composition of the present invention may be used in combination with one or more central nervous system drugs.

In the composition of the present invention, donepezil and sildenafil may be single-administered as pure compounds in a single dose or multiple doses, or may be administered together with pharmaceutically acceptable carriers or excipients. The pharmaceutical composition according to the present invention may be formulated not only with pharmaceutically acceptable carriers or diluents, but also with any other known adjuvants and excipients using conventional techniques as disclosed in the document (see, Remington: The Science and Practice of Pharmacy, 21 Edition, Hauber, Ed., Lippincott Williams & Wilkins, 2006).

For administration, the pharmaceutical composition of the present invention may include a pharmaceutically acceptable carrier, excipient and/or diluent, and the like. The carrier, excipient and/or diluent may include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil, but the present invention is not limited thereto.

The pharmaceutical composition of the present invention may be prepared in a pharmaceutical formulation using methods well known in the art. In the preparation of the formulation, the active ingredient may be mixed or diluted with a carrier, or encapsulated in a carrier in the form of a container. When the pharmaceutical composition of the present invention is prepared in a formulation for oral administration, for example, the pharmaceutical composition may be formulated into tablets, troches, lozenges, aqueous or oily suspensions, prepared powders or granules, emulsions, hard or soft capsules, syrups or elixirs.

In an aspect, the present invention provides a method for preventing or treating Alzheimer's disease or cognitive impairment, including administering to a patient a therapeutically effective amount of donepezil and sildenafil. In another aspect, the present invention provides the use of donepezil and sildenafil in the preparation of a medicament for preventing or treating Alzheimer's disease or cognitive impairment. The donepezil, sildenafil, salt, and the like, are the same as described above.

In an aspect, the present invention provides a combination for preventing, treating or improving Alzheimer's disease or a cognitive impairment disease, including a first agent including donepezil and a second agent including sildenafil.

In the present invention, the term combination means a combination of two or more active substances in a formulation and a combination in the sense of separate formulations of the active substances to be administered at specified intervals from one another in treatment. Thus, the term combination, when described in the context of the present invention, includes the clinical realization of the co-administration of two or more therapeutically effective compounds.

In the combination of the present invention, the first agent and/or the second agent may be administered parenterally or orally, respectively, preferably orally.

In the combination of the present invention, the first agent and the second agent may be administered simultaneously or at different times.

The combination of the present invention may be a combination formulation including the first agent and the second agent, specifically, a combination formulation administered orally.

In an aspect, the present invention provides an adjuvant composition of donepezil for improving Alzheimer's disease or cognitive impairment, including sildenafil or a pharmaceutically acceptable salt thereof.

In the present invention, the term adjuvant means the use that even though the prevention, treatment or improvement effect of a drug alone administered as an adjuvant is relatively low, the effect of preventing, treating or improving Alzheimer's disease or cognitive impairment is remarkably improved when administered in combination with other central nervous system drugs.

The composition of the present invention may be a pharmaceutical composition or a food composition. When the composition is used as a food composition, donepezil, sildenafil, or a pharmaceutically acceptable salt thereof may be added as it is or may be used together with other foods or food ingredients, and may be appropriately used according to a conventional method. In addition to the active ingredient, the composition may include food supplementary additives that are acceptable for food, and the mixing amount of the active ingredient may be suitably determined according to the purpose of use (prophylactic, health or therapeutic treatment).

The food composition of the present invention may include a health functional food. The term “health functional food” used in the present invention refers to food manufactured and processed in the form of tablets, capsules, powders, granules, liquids, and pills, and the like, using raw materials or ingredients having useful functions for the human body. The term “functional” means to obtain a useful effect for health purposes, such as regulation of nutrients, physiological action, or the like, on the structure and function of the human body.

In addition, there is no limitation on the type of health food in which the composition of the present invention is capable of being used. Further, the composition including donepezil and/or sildenafil of the present invention as an active ingredient may be prepared by mixing other appropriate auxiliary ingredients that may be contained in a health functional food and known additives according to the selection of those skilled in the art. Examples of food capable of being added may include meat, sausage, bread, chocolate, candy, snacks, confectionery, pizza, ramen, other noodles, gum, dairy products including ice cream, various soups, beverages, tea, drinks, alcoholic beverages and vitamin complexes, and the like, and the food may be prepared by adding the extract prepared according to the present invention as a main component to juice, tea, jelly, juice, and the like.

Matters mentioned in all compositions, methods of treatment and uses of the present invention are applied in the same way as long as they do not contradict each other.

The present invention will be described in more detail through the following Examples. However, the following Examples are only described for specifying the contents of the present invention, and the present invention is not limited thereto.

### <Example 1> Confirmation of Synaptic Plasticity Synergistic Effect Depending on the Combined Administration of Donepezil and Sildenafil in Animal Model of Alzheimer's Disease or Cognitive Impairment

To confirm synaptic plasticity by the combined administration of donepezil and sildenafil, mice were divided into (1) Vehicle group used as control (n=4), (2) amyloid beta group (n=4), (3) amyloid beta+donepezil (100 nM) single treatment group (n=4), and (4)-(8) amyloid beta+donepezil (100 nM)+sildenafil (50, 75, 100, 150, 200 nM) combination treatment group (each group n=4), and long-term potentiation (LTP) was recorded from brain slices in each group.

Neurons in the brain transfer information through junctions called synapses. A series of processes in which synapses are strengthened or weakened (synaptic plasticity) is an important process for learning and memory. The continuous improvement in the size and activity of a synapse is called synaptic long-term potentiation (LTP), which is a representative form of synaptic plasticity. The LTP is an important mechanism of higher cognitive function, and synaptic plasticity impairments including LTP deficit are common in dementia.

In order to confirm an effect of donepezil and combined administration according to the present invention on the treatment of Alzheimer's disease or cognitive impairment, changes in synaptic long-term potentiation (LTP) were evaluated in brain slices of a mouse model of the Alzheimer's disease.

Specifically, in order to construct a model of Alzheimer's disease or cognitive impairment, a mouse brain slice was treated with 3 μM amyloid beta (Aβ) which is a major component of amyloid plaques found in the brain of Alzheimer's patients. Amyloid beta (Aβ) is known as a major component of cognitive impairment.

As a result of the experiment, it was confirmed that LTP was significantly improved in the donepezil and sildenafil combination treatment group as compared to the donepezil single treatment group. In particular, as a result of comparing and measuring the LTP effect depending on various weight ratios of donepezil and sildenafil, it was confirmed that the best effect was obtained in the range where the weight ratio of donepezil and sildenafil was 1:0.6 to 1:2.0 (Table 1 or FIG. 1).

TABLE 1

| | | Amyloid beta | | | | | |
|---|---|---|---|---|---|---|---|
| | | | Donepezil 100 nM | | | | |
| | Amyloid | Donepezil | Sildenafil | Sildenafil | Sildenafil | Sildenafil | Sildenafil |
| | beta | 100 nM | 50 nM | 75 nM | 100 nM | 150 nM | 200 nM |
| Synaptic | 0.00 | 9.18 | 7.76 | 14.25 | 29.77 | 18.76 | 10.21 |
| long-term | | | | | | | |
| potentiation | | | | | | | |
| (Increase | | | | | | | |
| or decrease | | | | | | | |
| compared to | | | | | | | |
| Amyloid | | | | | | | |
| beta group) | | | | | | | |

The data in the above table indicate that synaptic long-term enhancement was increased by 9.18% in the group treated with donepezil alone and 29.77% in the group treated with donepezil and sildenafil (sildenafil 100 nM), based on the amyloid beta group.

This indicates that the donepezil and sildenafil complex agent according to the present invention improved the therapeutic effect on Alzheimer's disease or cognitive impairment, as compared to donepezil, in the synaptic long-term potentiation process of the hippocampus, which is known to be deeply involved in the mechanism of memory.

### <Example 2> Confirmation of Improvement Effect on Cognitive Function Through Morris Water Maze According to the Combined Administration of Donepezil and Sildenafil in Animal Model of Alzheimer's Disease or Cognitive Impairment

The 5×FAD mouse used in this experiment was a genetically modified mouse model in which Alzheimer's disease-inducing genes were inserted. Specifically, mice over-expressed both the amyloid precursor protein with the Swedish mutation (K670N, M671L), the Florida mutation (I716V), and the London mutation (V717I) and PS1 with the M146L and L286V mutations. The 5×FAD mouse model is the most widely used in Alzheimer's disease studies.

A Morris water maze (MWM) task was conducted to examine an effect on improving cognitive function depending on the administration of the complex composition of the present invention. The experimental equipment was comprised of a circular water tank (150 cm in diameter and 45 cm in height) filled with water having a temperature of about 22±2° C. an escape platform (10 cm in diameter and 30 cm in height), and 4 markers attached to the wall that allows the mouse to remember the location of the escape platform. The above markers remained unchanged throughout the test period. The escape platform was placed 1 cm above the water surface or 0.5 to 1.5 cm below the water surface according to the purpose of the experiment. When placed under the water surface, the water was made opaque with white water-based paint so that the escape platform was not visible to the eye. The Morris water maze was divided into four quadrants, i.e., northeast (NE), northwest (NW), southeast (SE) and southwest (SW), wherein the escape platform was placed in the center of the southwest quadrant, and the mouse started randomly from one of the others. The location of the escape platform did not change throughout the learning period.

The escape platform was made visible on Learning DAY 0. Each mouse was trained to recognize that it was possible to escape when they swam toward the escape platform and landed thereon. On DAY 1-4, the escape platform was hidden by being placed under the water, and then the mice learned the position of the escape platform. The time limit was set to 60 seconds, and if the mouse found the escape platform within seconds, it was allowed to stay on the escape platform for 5 seconds. The mice were then immediately transferred to the breeding cage. On DAY 5, the probe test was performed without the escape platform. The probe test was performed by placing the mouse in the farthest part of the quadrant where the escape platform was placed during the training day, and 60 seconds was set as the time limit. The indexes of cognitive function include the escape latency which is the time required for the mouse to find the escape platform and the time spent in the quadrant where the escape platform was placed. It was judged that the shorter the time it takes to escape (FIG. 2A) and the longer the mouse stays in the quadrant where the escape platform was placed (FIG. 2B), the better the memory and learning ability of the mouse.

In order to confirm the improvement effect on cognitive impairment by the combined administration of donepezil and sildenafil, the mice were divided into the following groups: (1) normal mouse vehicle (shown as WT vehicle, 5 ml/kg of methyl cellulose, n=5), (2) mutated mouse (shown as MT vehicle, 5 ml/kg of 0.5% methylcellulose, n=5), (3) MT donepezil (2 mg/kg of single administration, n=4), and (4)-(5) MT donepezil+sildenafil (combined administration of 1 mg/kg of donepezil+2 mg/kg of sildenafil, n=5, and combined administration of 2 mg/kg of donepezil+2 mg/kg of sildenafil, n=5) groups. Then, each group was subjected to oral administration for 4 weeks to perform the experiment.

As a result, it was shown that when compared with the mutant mouse vehicle administration group in the escape latency on the last day of learning (DAY 4), the donepezil and sildenafil combined administration group showed significantly reduced escape latency time as compared to the 2 mg/kg of donepezil single administration mutant mouse groups (Table 2 or FIG. 2A).

TABLE 2

| | Mutated mouse | | | |
|---|---|---|---|---|
| | | | Donepezil | Donepezil |
| | | | 1 mg/kg | 2 mg/kg |
| | | Donepezil | Sildenafil | Sildenafil |
| | Vehicle | 2 mg/kg | 2 mg/kg | 2 mg/kg |
| Escape latency | 0.00 | −14.2 | −41.52 | −56.28 |
| (Increase or | | | | |
| decrease compared | | | | |
| to mutated mouse | | | | |
| vehicle group) | | | | |

The data in the above table shows that the escape latency was reduced by 14.2% in the group administered with 2 mg/kg of donepezil alone, and by 56.28% in the group administered with 2 mg/kg of donepezil and 2 mg/kg of sildenafil, based on the mutant mouse vehicle group.

In addition, it was confirmed that the time spent in the quadrant was significantly increased in the donepezil and sildenafil combined administration group (2 mg/kg of donepezil and 2 mg/kg of sildenafil) as compared to the donepezil single administration mutant mouse groups (Table 3 or FIG. 2B).

TABLE 3

| | Mutated mouse | | | |
|---|---|---|---|---|
| | | | donepezil | donepezil |
| | | | 1 mg/kg | 2 mg/kg |
| | | donepezil | sildenafil | sildenafil |
| | vehicle | 2 mg/kg | 2 mg/kg | 2 mg/kg |
| time spent in the | 0.00 | 73.20 | 93.78 | 153.78 |
| target quadrant | | | | |
| (Increase or decrease | | | | |
| compared to mutated | | | | |
| mouse vehicle group) | | | | |

The data in the above table show that the time spent in the target quadrant was increased by 73.20% in the group administered with 2 mg/kg of donepezil alone, and 153.78% in the group administered with 2 mg/kg of donepezil and 2 mg/kg of sildenafil, based on the mutant mouse vehicle group.

This indicates that the combined administration of donepezil and sildenafil significantly improved memory and learning ability, as compared to the improvement effect obtained by single administration of donepezil. In particular, it was reconfirmed that the improvement effect on cognitive function was maximized in the range where the weight ratio of donepezil and sildenafil was 1:0.6 to 1:2.0 among the combined administration groups as confirmed in FIG. 1.

1. A composition for preventing, treating or improving Alzheimer's disease or cognitive impairment comprising:
(i) donepezil or a pharmaceutically acceptable salt thereof; and
(ii) sildenafil or a pharmaceutically acceptable salt thereof.

2. The composition of claim 1, wherein a weight ratio of (i) donepezil or the pharmaceutically acceptable salt thereof; and (ii) sildenafil or the pharmaceutically acceptable salt thereof is 1:0.6 to 1:2.0.

3. The composition of claim 1, wherein (i) donepezil or the pharmaceutically acceptable salt thereof; and (ii) sildenafil or the pharmaceutically acceptable salt thereof are administered simultaneously or at different times.

4. The composition of claim 1, wherein the composition is in a combination formulation including (i) donepezil or a pharmaceutically acceptable salt thereof; and (ii) sildenafil or a pharmaceutically acceptable salt thereof.

5. The composition of claim 4, wherein the combination formulation includes (i) 2.5 to 23 mg of donepezil or the pharmaceutically acceptable salt thereof; and (ii) 2.5 to 50 mg of sildenafil or the pharmaceutically acceptable salt thereof.

6. The composition of claim 1, wherein the composition is a pharmaceutical composition or a food composition.

7. A composition for improving cognitive function comprising:
(i) donepezil or a pharmaceutically acceptable salt thereof; and
(ii) sildenafil or a pharmaceutically acceptable salt thereof.

8. The composition of claim 7, wherein a weight ratio of (i) donepezil or the pharmaceutically acceptable salt thereof and (ii) sildenafil or the pharmaceutically acceptable salt thereof is 1:0.6 to 1:2.0.

9. A method for preventing, treating or improving Alzheimer's disease or cognitive impairment, the method comprising:
administering the composition of claim 1 to a subject in need thereof.

10. A method for improving cognitive function, the method comprising:
administering the composition of claim 7 to a subject in need thereof.
